SEQUENCE LISTING (13 sequences)

SEQ ID NO: 1 - protein, 5 aa, Artificial
  Biopolymer pentapeptide repeat sequence
VPGXG

SEQ ID NO: 2 - protein, 4 aa, Artificial
  Biopolymer tetrapeptide repeat sequence
VPGG

SEQ ID NO: 3 - protein, 4 aa, Artificial
  Biopolymer tetrapeptide repeat sequence
GGVP

SEQ ID NO: 4 - protein, 4 aa, Artificial
  Biopolymer tetrapeptide repeat sequence
GGFP

SEQ ID NO: 5 - protein, 4 aa, Artificial
  Biopolymer tetrapeptide repeat sequence
GGAP

SEQ ID NO: 6 - protein, 5 aa, Artificial
  Biopolymer pentapeptide repeat sequence
VPGVG

SEQ ID NO: 7 - protein, 5 aa, Artificial
  Biopolymer pentapeptide repeat sequence
GVGVP

SEQ ID NO: 8 - protein, 5 aa, Artificial
  Biopolymer pentapeptide repeat sequence
GKGVP

SEQ ID NO: 9 - protein, 5 aa, Artificial
  Biopolymer pentapeptide repeat sequence
GVGFP

SEQ ID NO: 10 - protein, 5 aa, Artificial
  Biopolymer pentapeptide repeat sequence
GFGFP

SEQ ID NO: 11 - protein, 5 aa, Artificial
  Biopolymer pentapeptide repeat sequence
GEGVP

SEQ ID NO: 12 - protein, 5 aa, Artificial
  Biopolymer pentapeptide repeat sequence
GFGVP

SEQ ID NO: 13 - protein, 5 aa, Artificial
  Biopolymer pentapeptide repeat sequence
GVGIP

(19) United States
(12) Patent Application Publication
(10) Pub. No.: US 2010/0189643 A1
(43) Pub. Date: Jul. 29, 2010

(54) DRUG DELIVERY WITH STIMULUS RESPONSIVE BIOPOLYMERS

(75) Inventors: Ashutosh Chilkoti, Durham, NC (US); Matthew Robert Dreher, Durham, NC (US); Daniel Eugene Meyer, Durham, NC (US)

Correspondence Address: COOLEY LLP;ATTN: Patent Group, Suite 1100, 777 - 6th Street, NW, WASHINGTON, DC, 20001, US

(73) Assignee: DUKE UNIVERSITY, Durham, NC (US)

(21) Appl. No.: 12/374,816

(22) PCT Filed: Jul. 17, 2007
(86) PCT No.: PCT/US2007/016152

§ 371 Date: Apr. 8, 2010

Related U.S. Application Data
(60) Provisional application No. 60/832,817, filed on Jul. 24, 2006.

(51) Int. Cl. A61K 47/48; A61K 49/00; A61K 51/00

(52) U.S. Cl. 424/1.65; 530/402; 530/391.7; 424/9.1

(57) ABSTRACT

The present invention provides conjugate compounds comprising (a) an active compound; (b) optionally, but in some embodiments preferably, an affinity binding agent; and (c) a block copolymer, the block copolymer comprising: (i) a first elastin-like polypeptide having a first Tt and (U) a second elastin-like polypeptide having a second Tt greater than the first Tt. Method for the targeted delivering of an active compound in vivo to a selected region within a subject with such agents are also described.

This application claims benefit of U.S. Provisional Patent Application Ser. No. 60/832,817, filed Jul. 24, 2006, the content of which is incorporated herein by reference in its entirety.

This invention was made with Government support under grant number EB00188 and GM-061232 from the National Institutes of Health. The US Government has certain rights to this invention.

### BACKGROUND OF THE INVENTION

Cancer describes a collection of diseases caused by multiple genetic mutations arising from environmental insults, somatic DNA replication error and inherited genetic defects. In the United States, cancer is the second leading cause of death with approximately half of all men and one-third of all women developing cancer in their lifetime, resulting in an annual cost of about 170 billion dollars. The cancer phenotype is characterized by uncontrolled growth of abnormal cells with limitless replicative potential and invasion of surrounding tissues. Eighty-five percent of cancer patients have solid tumors and 50% of those patients die as a result of malignant disease. Although metastases are often the ultimate cause of death, a critical failure in therapy, ultimately leading to metastases, is due to the lack of control of the primary tumor. Local control of the primary tumor is particularly difficult in the cervix, colon, ovarian, pancreas, and brain. There is hence an urgent need to improve therapy of primary tumors.

The modern treatment of cancer typically includes various combinations of external beam radiation, chemotherapy, surgery or experimental methods. In general, both radiation and chemotherapy derive their therapeutic efficacy from selective toxicity to rapidly proliferating cells. However, cancer cells are not the only rapidly proliferating cells in the body and toxic side effects are commonly found in hematopoietic progenitor cells of the bone marrow and epithelial cells of the gut. Surgery involves the excision of the tumor mass itself, which has limited effects on normal surrounding cells, but tumor margins can be difficult to define and micrometastases are too small to be surgically removed (Tannock & Hill, eds. (1998) The Basic Science of Oncology. 3^{rd} ed., McGraw-Hill: New York). Experimental methods including immunotherapy (Carter (2001) Nat. Rev. Canc. 1(2):118-129), gene therapy (McCormick (2001) Nat. Rev. Canc. 1(2):130-141) and hyperthermia (Dewhirst, et al. (1997) Semin. Oncol. 24(6):616-625) have shown some promise but require additional investigation to ascertain their potential widespread benefit.

The goal of drug delivery in cancer therapy is to increase the concentration of a therapeutic agent within the tumor and also limit systemic exposure. Numerous drug delivery technologies have been developed to accomplish this goal, including liposomes (Sharma & Sharma (1997) Internat. J. Pharmaceut. 154(2):123-140), micelles (Kataoka, et al. (2001) Adv. Drug Deliv. Rev. 47(1):113-131), antibody-directed enzyme-prodrug therapy (Senter & Springer (2001) Adv. Drug Deliv. Rev. 53(3):247-264), photodynamic therapy (Vrouenraets, et al. (2003) Anticancer Res. 23(1B):505-522), affinity targeting (Allen (2002) Nat. Rev. Canc. 2(10):750-763) and macromolecular drug carriers (Duncan (2003) Nat. Rev. Drug Discov. 2(5):347-360). In general, a therapeutic agent's toxicity is proportional to the exposure of a cell to that agent. Therefore, the therapeutic efficacy of drug delivery is gained through increasing the concentration of the drug in the tumor relative to normal tissues.

Many of these drug delivery modalities described above take advantage of the unique pathophysiology of tumor vasculature. Tumors contain a high density of abnormal blood vessels that lack proper differentiation with dilated chaotic architecture and aberrant branching (Algire, et al. (1945) J. Natl. Canc. Inst. 6:73-85; Ide, et al. (1939) Am. J. Roentgenol. 42:891-899; Lewis (1927) Bull. Johns Hopkins Hospital 41:156-162; Sandison (1928) Am. J. Anat. 41:475-496). The tumor vasculature also has impaired function, such as increased vascular permeability that contributes to the greater accumulation of plasma proteins in a tumor as compared with normal tissues (Babson & Winnick (1954) Cancer Res. 14:606-611; Busch & Greene (1955) Yale J. Biol. Med. 27:339-349; Dewey (1959) Am. J. Physiol. 197:423-431; Duran-Reynals, (1939) Am. J. Cancer 35:98-107; Dvorak, et al. (1988) Am. J. Pathol. 133(1):95-109; Gerlowski & Jain (1986) Microvasc. Res. 31(3):288-305; Heuser & Miller (1986) Cancer 57(3):461-464; Peterson & Appelgren (1973) Eur. J. Cancer 9:543-547; Song & Levitt (1971) Cancer Res. 31:587-589; Underwood & Carr (1972) J. Pathol. 107:157-166). This phenomenon was elucidated by Maeda and coworkers, who described it as the “enhanced permeability and retention” (EPR) effect (Maeda & Matsumura (1989) Crit. Rev. Therapeut. Drug Carrier Systems 6(3):193-210; Matsumura & Maeda (1986) Cancer Res. 46(12):6387-6392; Seymour (1992) Crit. Rev. Therapeut. Drug Carrier Systems 9(2):135-187), which combines the increased permeability of tumors with a slower rate of clearance due to the lack of functional lymphatics, thereby resulting in the increased accumulation of macromolecules and nanoparticles (<100 nm) in tumors. These findings strongly advocate the use of macromolecules and nanoparticles for tumor diagnosis and therapy as drug carriers.

Macromolecular drug carriers encompass large molecules that are typically linked to a therapeutic agent and target solid tumors either “passively,” based on the EPR effect, or “actively,” due to a specific affinity or stimulus (Duncan (2003) Nat. Rev. Drug Discov. 2(5):347-360; Ringsdorf (1975) J. Poly. Sci. 51:135-153; Tomlinson (1985) J. Controlled Rel. 2:385-391). In addition to the EPR effect, macromolecular drug carriers are attractive for drug delivery because they have longer plasma half-lives, reduced normal tissue toxicity, activity against multiple drug-resistant cell lines and the ability to increase the solubility of poorly soluble drugs (Duncan (1992) Anti-Cancer Drugs 3(3):175-210; Duncan, et al. (1998) Hum. Exp. Toxicol. 17(2):93-104; Ohkawa, et al. (1993) Cancer Res. 53(18):4238-4242; Ryser & Shen (1978) Proc. Natl. Acad. Sci. USA 75(8):3867-3870; Seymour, et al. (1987) Cancer Treat. Rev. 14(3-4):319-327; St'astny, et al. (1999) Euro. J. Cancer 35(3):459-466; Takakura, et al. (1994) Intl. J. Pharmac. 105(1):19-29; Yamaoka, et al. (1994) J. Pharmac. Sci. 83(4):601-606; Yeung, et al. (1991) Cancer Chemo. Pharmacol. 29(2):105-111). These attributes often result in higher anticancer efficacy for passive macromolecular drug carriers as compared to low molecular weight drugs (Duncan (2003) Nat. Rev. Drug Discov. 2(5):347-360; Cassidy, et al. (1989) Biochem. Pharmacol. 38(6):875-879; Ghandehari & Cappello (1998) Pharmac. Res. 15(6):813-815; Haider, et al. (2004) J. Contr. Rel. 95(1):1-26; Kabanov, et al. (2002) J. Contr. Rel. 82(2-3):189-212; Kabanov, et al. (2005) J. Contr. Rel. 101(1-3):259-271; Kopecek (2003) Euro. J. Pharmac. Sci. 20(1):1-16; Kopecek, et al. (2000) Eur. J. Pharm. Biopharm. 50(1):61-81; Li, et al. (2003) J. Biomed. Mater. Res. A 65A(2):196-202; Lukyanov & Torchilin (2004) Adv. Drug Deliv. Rev. 56(9):1273-1289; Megeed, et al. (2002) Adv. Drug Deliv. Rev. 54(8):1075-1091; Minko, et al. (2000) Intl. J. Cancer 86(1):108-117; Minko, et al. (1998) J. Contr. Rel. 54(2):223-233; Mitra, et al. (2005) J. Contr. Rel. 102(1):191-201; Nagarsekar & Ghandehari (1999) J. Drug Target. 7(1):11-32; Nan, et al. (2005) J. Drug Target. 13(3):189-197; Roy, et al. (1999) Nat. Med. 5(4):387-391; Shiah, et al. (1999) J. Contr. Rel. 61(1-2):145-157; Torchilin (2001) J. Contr. Rel. 73(2-3):137-172; Torchilin, et al. (2003) Proc. Natl. Acad. Sci. USA 100(10):6039-6044; Wen, et al. (2003) J. Contr. Rel. 92(1-2):39-48; Whiteman, et al. (2001) J. Lipos. Res. 11(2-3):153-164). The most compelling evidence for the advantages of using polymer-drug conjugates over free chemotherapeutic agents for the treatment of cancer come from extensive preclinical and clinical studies by Kopecek and colleagues on the use of poly N-(2-hydroxypropyl) methacrylamide (polyHPMA) copolymers as drug carriers (Kopecek, et al. (2000) Eur. J. Pharm. Biopharm. 50(1):61-81, and references within). Since this phenomenon was first reported in 1986, there have been at least nine clinical trials investigating polymeric macromolecular drug carriers (Duncan (2003) Nat. Rev. Drug Discov. 2(5):347-360) with the intention of exploiting the EPR effect. Other drug delivery systems based on self-assembly, such as liposomes and nano-particles, also take advantage of the EPR effect.

In an aqueous environment, block-copolymers exhibiting amphiphilic characteristics and a large solubility difference between blocks will assemble into micelles composed of a hydrophobic core surrounded by a hydrophilic corona. These core-shell structures exhibit a narrow size distribution in the range of tens of nanometers, have controllable properties such as block type and hydrophobicity, as well as the ability to present functional groups both in the core and corona, and to solubilize otherwise hydrophobic drugs, thereby sequestering them from the solution environment. Due to these desirable properties, significant effort has been devoted to the design of polymeric micelles as drug carriers for cancer therapy (Kataoka, et al. (1993) J. Contr. Rel. 24:119-132). In addition, the mesoscopic size of polymeric micelles is particularly attractive for applications using the EPR effect (Matsumura & Maeda (1986) Cancer Res. 46(12):6387-6392), while the hydrophilic corona prevents uptake into the reticuloendothelial system (RES) (Ishida, et al. (1999) Intl. J. Pharma. 190(1):49-56).

A functional micelle must be water soluble, show sufficient stability both in vitro and in vivo, incorporate an active amount of drug, have a reasonable biological half-life, and decompose into biologically safe byproducts (Allen, et al. (1999) Colloids Surf. B: Biointerfaces 16:1-35; Lavasanifar, et al. (2002) Adv. Drug Deliv. Rev. 54(2):169-190). Synthetic polymers such as poly-ethylene glycol and poly-ethylene oxide have been used as the hydrophilic block in order to limit the interaction with foreign bodies that may reduce the plasma half-life (Kwon & Kataoka (1995) Adv. Drug Deliv. Rev. 16(2-3):295-309). Other block copolymers contain both synthetic and biomimetic polymers to optimize the micelle behavior with regards to the above criteria (Kataoka, et al. (2001) Adv. Drug Deliv. Rev. 47(1):113-131). Polymeric micelles are gaining increasing credibility as drug carriers, in particular given the increase in efficacy of doxorubicin/adriamycin observed when encapsulated in micellar form (Kwon, et al. (1995) Pharm. Res. 12(2):192-195; Kwon, et al. (1997) J. Contr. Rel. 48(2-3):195-201) and taxol (Torchilin, et al. (2003) Proc. Natl. Acad. Sci. USA 100(10):6039-6044). Kataoka's original PEG-PLBA/DOX micelle system (Matsumura, et al. (2004) Br. J. Cancer 91(10):1775-1781) is now in clinical trials so that the interest in using polymeric micelles for cancer therapy should continue to grow.

The next generation of “smart”, environmentally responsive, polymeric micelles has appeared in the literature. For example, pH-sensitive micelles have been created to release a conjugated drug in lysosomal compartment of the cell (Bae, et al. (2005) Bioconj. Chem. 16(1):122-130). Thermosensitive block copolymers composed of pNIPAAM or Elastin-like Polypeptide (ELP) blocks with distinct transition temperatures have both been shown to form micelle and aggregate structures over a temperature range spanning the transition temperatures of each block (Chung, et al. (2000) J. Contr. Rel. 65(1-2):93-103; Meyer & Chilkoti (2002) Biomacromolecules 3(2):357-367). Furthermore, pNIPAAM has been shown to release hydrophobic compounds following the transition from a well-organized micelle to lesser-defined aggregate (Chung, et. al. (1999) J. Contr. Rel. 62(1-2):115-127).

Another development in the design of polymer micelles is the incorporation of targeting ligands to the ends of the coronal segments in order to create multivalent nanoparticles; the presented ligands include antibodies (Torchilin (2001) J. Contr. Rel. 73(2-3):137-172), folic acid (Kim, et al. (2005) J. Contr. Rel. 103:625-634), and peptide ligands such as the Asp-Gly-Arg (RGD) motif (Nasongkla, et al. (2004) Angewandte Chemie-International Ed. 43(46):6323-6327). Despite these studies on stimulus-responsive micelles and receptor-targeted micelles, the concept of thermally triggered self-assembly of micelles only in tumors for selective polyvalent targeting of tumors has not been suggested in the prior art.

One method to augment therapy and drug transport is to heat the tumor with mild hyperthermia. Temperatures in a tumor during a hyperthermia treatment typically range from 39-45° C. and treatment durations are between 60 and 120 minutes (Dewhirst (1995) In Thermo-radiotherapy and thermochemotherapy, Seegenschmiedt, et al. (Eds.) Springer-Verlag: Berlin. pg. 123-136). Hyperthermia has a marked effect at every biological level including decreased DNA synthesis, altered protein synthesis such as induction of heat shock proteins, disruption of the microtubule organizing center, altered expression of receptors and binding of growth factors, and changes in cell morphology and attachment are some of the many effects observed at the subcellular and cellular levels. Furthermore, hyperthermia is known to increase tumor blood flow and vascular permeability (Dewhirst, et al. (1997) Semin. Oncol. 24(6):616-625; Dewhirst, et al. (1989) Intl. J. Rad. Oncol. Biol. Phys. 17(1):91-99; Engin (1996) Control. Clin. Trials 17(4):316-342; Vaupel, et al. (1988) Intl. J. Hyperthermia 4(3):307-321). The combination of these biological and physiological effects result in the increased efficacy in the treatment of cancer when used as an adjuvant therapy with radiation and chemotherapy (Dewhirst, et al. (1997) Semin. Oncol. 24(6):616-625; Wust, et al. (2002) Lancet Oncol. 3(8):487-97). Several external methods are clinically available for localized, internal heating of targeted regions, including radio-frequency, microwave, or focused ultrasound beams (Feyerabend, et al. (1997) Anticancer Res. 17(4B):2895-2897).

Elastin-like Polypeptides are temperature-sensitive biopolymers composed of a Val-Pro-Gly-Xaa-Gly (SEQ ID NO:1) pentapeptide repeat (where the Xaa is any amino acid residue except Pro) derived from a structural motif found in mammalian elastin (Gray, et al. (1973) Nature 246(5434):461-466; Tatham & Shewry (2000) Trends Biochem. Sci. 25(11):567-571). ELPs undergo a sharp inverse temperature phase transition, also called a lower critical solution temperature transition, in response to an increase in temperature (Urry (1992) Prog. Biophys. Mol. Biol. 57(1):23-57; Urry (1997) J. Phys. Chem. B 101(51):11007-11028; Li, et al. (2001) J. Am. Chem. Soc. 123(48):11991-11998). ELPs are soluble in aqueous solutions at temperatures below their transition temperature (Tₜ) but become insoluble and aggregate at temperatures above their Tₜ. The inverse temperature transition is fully reversible, such that the aggregated ELP becomes soluble when the temperature is decreased below its Tₜ. The Tₜ can be tuned by adjusting the identity of the Xaa residue, molecular weight and ELP concentration.

Classical immunotargeting or immunotherapy of tumors was first proposed by Ehrlich ((1906) Collected Studies on Immunity. 1^{st} Ed. New York: John. Wiley and Sons) who envisioned “magic bullets” that could selectively deliver therapeutic agents directly to the cancer cell. By 2001 there were at least 15 ongoing clinical trials using antibody-based therapies and five FDA-approved antibodies for the treatment of cancer (Carter (2001) Nat. Rev. Canc. 1(2):118-129). Affinity-targeted drug delivery involves the delivery of a therapeutic molecule to a tumor through recognition of tumor antigens or receptors (Allen (2002) Nat. Rev. Canc. 2(10):750-763). In the most general approach, a therapeutic agent is attached to a targeting molecule and administered intravenously. The targeting molecule may not only be an antibody or fragment thereof (Carter (2001) Nat. Rev. Canc. 1(2):118-129; Bast, et al. (2000) Cancer Medicine. 5^{th} ed; Wikstrand, et al. (1999) Cancer Metast. Rev. 18(4):451-464), but also peptides (Arap, et al. (1998) Science 279(5349):377-380) and small molecules (Lu & Low (2002) Cancer Immun. Immunother. 51(3):153-162). The antigens recognized by a targeting molecule are either tumor-specific or tumor-associated. Tumor-specific antigens are only presented on tumor cells and arise from either somatic mutations or tumor viruses. A tumor-specific antigen (e.g., cancer-testis antigen) is an excellent target because it is not presented on normal tissue; however, these antigens tend to be isolated to a particular cancer and cannot be ubiquitously targeted in cancer treatment. In contrast, tumor-associated antigens (e.g., carcinoembryonic antigen, CEA) can be ubiquitously targeted in many cancer types since they arise from an over-expression of a characteristic cancer protein. The drawback of tumor-associated antigens is that they are also expressed on normal tissues although at lower levels, therefore reducing the specificity of this strategy (Stevanovic (2002) Nat. Rev. Cancer 2(7):514-520). Although affinity targeting is a promising approach, it has yet to bear this promise in the clinic and has met with varying degrees of success using current approaches (Allen (2002) Nat. Rev. Canc. 2(10):750-763).

### SUMMARY OF THE INVENTION

A first aspect of the present invention is a conjugate compound comprising, consisting of, or consisting essentially of:

(a) an active compound;

(b) optionally, but in some embodiments preferably, an affinity binding agent; and

(c) a block copolymer, the block copolymer comprising, consisting of or consisting essentially of: (i) a first elastin-like polypeptide having a first Tₜ and (ii) a second elastin-like polypeptide having a second Tₜ greater than the first Tₜ.

A further aspect of the invention is a method for the targeted delivering of an active compound in vivo to a selected region within a subject, comprising:

(a) administering a conjugate compound as described above to the subject wherein the affinity binding agent selectively binds to the selected region;

(b) heating the selected region to a temperature greater than the first Tₜ so that the active compound is preferentially localized in the selected region.

A further aspect of the invention is a composition comprising, consisting of or consisting essentially of a particle such as a micelle or vesicle in a carrier, the micelle or vesicle comprising, consisting of or consisting essentially of:

(a) an active compound;

(b) optionally, but in some embodiments preferably, an affinity binding agent; and

(c) a block copolymer, the block copolymer comprising, consisting of or consisting essentially of: (i) a first elastin-like polypeptide having a first Tₜ and (ii) a second elastin-like polypeptide having a second Tₜ greater than the first Tₜ.

A further aspect of the invention is a method for the targeted delivering of an active compound in vivo to a selected region within a subject, comprising:

(a) administering a composition as described above to the subject wherein the affinity binding agent selectively binds to the selected region;

(b) heating the selected region to a temperature greater than the first Tₜ so that the active compound is preferentially localized in the selected region.

Suitable active agents include but are not limited to imaging agents, contrast agents, therapeutic agents, and radionuclides. Suitable affinity binding agents include antibodies, peptides, and synthetic molecules.

Administering may be a systemic administering step, such as carried out by subcutaneous injection, intraperitoneal injection, intraveneous injection, intramuscular injection, oral administration, inhalation administration, or transdermal administration.

A goal of this invention is to selectively deliver active compounds of interest (such as anticancer drugs or imaging agents) to a region such as a solid tumor in order to improve therapeutic efficacy, limit systemic toxicity or gain clinically relevant information

In some embodiments the invention is a novel macromolecular drug carrier, consisting of elastin-like polypeptides (ELPs), which will target solid tumors thereby delivering active compounds of interest to the tumor. ELPs belong to a unique class of biopolymers that undergo an inverse temperature phase transition; they are soluble at temperatures below their transition temperature (Tₜ) but become insoluble and aggregate at temperatures above their Tₜ (Urry (1992) Prog. Biophys. Mol. Biol. 57(1):23-57; Urry (1997) J. Phys. Chem. B 101(51):11007-11028; Li, et al. (2001) J. Am. Chem. Soc. 123(48):11991-11998). A series of ELP block copolymers (ELP-BCs) are disclosed which were constructed by combining an ELP gene having a low Tₜ (ELP4) with an ELP gene that has a much higher Tₜ (ELP2). These ELP-BCs function as triggerable amphiphiles where they are highly soluble in aqueous solutions at low temperatures, but self-assemble and form spherical micelles or vesicles at temperatures between the Tₜ of both ELP blocks. As disclosed herein, there are numerous ways in which ELP-BC technology can be used for drug delivery or delivery of imaging agents.

### BRIEF DESCRIPTION OF THE DRAWINGS

FIG. 1. Schematic of ELP-BCs. An ELP2 and ELP4 gene are seamlessly fused together to create and ELP-BC. When the size and ratio of the blocks are correctly selected, the ELP-BC will self-assemble into a spherical micelle at 37° C.

FIG. 2. Temperature-dependent self-assembly and cryo-TEM of an ELP-BC. FIG. 2A, DLS and UV-vis spectrophotometry show that ELP2-96,4-60 (25 μM) forms a micelle at temperatures between the Tₜ of both ELP blocks. FIG. 2B, Spherical micelles were confirmed by cryo-TEM of ELP2-64,4-90 at a temperature that induces micelle formation.

FIG. 3. Dependence of pyrene fluorescence on temperature (FIG. 3A) and concentration (FIG. 3B) for 25 μM ELP2-64,4-90 in PBS. FIG. 3A, I₁/I₃ of pyrene decreased from 20-60° C. indicating a reduction in the polarity of the ELP2-64,4-90 solution. As pyrene partitions into the hydrophobic core, the ratio decrease. There was a pronounced decrease in I₁/I₃ at temperatures above the critical micelle temperature and below the second transition. FIG. 3B, I₁/I₃ at the inflection point of each temperature scan (as shown in FIG. 3A) was plotted as a function of ELP2-64,4-90 concentration. The inflection point of a sigmoid fit (solid line) was defined as the critical micelle concentration (CMC). Data are the mean±SD in FIG. 3B (n=3).

FIG. 4. Biodistribution and immunofluorescence 24 hours after iv administration of anti-Her2/neu single-chain antibody fragment (scFv). FIG. 4A, Biodistribution of anti-Her2/neu scFv±SEM with a range of affinities. The symbols are as follows; tumor is an open triangle, blood is a solid square, liver is an open square, muscle is an open diamond and spleen is an open circle. FIGS. 4B and 4C, Blood vessels were labeled with anti-CD31 Monoclonal antibody (light areas) and the scFv was labeled with anti-C6.5 scFv antiserum (filled areas with vertical lines). FIGS. 4B and 4C are representative of a scFv with a K_{D} of 10⁻⁷ and a scFv with a K_{D} of 10⁻¹¹, respectively. Note the diffuse fluorescent pattern of the low affinity scFv in contrast to the intense perivascular fluorescence of the high affinity scFv (original magnification 40×). Figure compiled from reference (Adams, et al. (2001) Cancer Res. 61(12):4750-4755).

FIG. 5. Pharmacokinetic analysis of [¹⁴C]ELP in mice (Balb/c nu/nu) reveals a characteristic distribution and elimination response after intravenous administration. The plasma concentration-time course was analyzed with a standard two-compartment pharmacokinetic model to approximate both distribution and elimination of the ELP. The data are presented as mean±standard deviation, n=5.

FIG. 6. Images of normal (FIG. 6A) and tumor vasculature (FIGS. 6B and 6C) from the dorsal window chamber and LSCM. In FIGS. 6A and 6B, the vasculature is visualized by intravenous injection of fluorescein-labeled 2 MDa dextran. Bright fluorescence was emitted from the lumen of the vessel because the images where taken shortly after injection. In FIG. 6C, the tumor was heated to 42° C., which is above the T, of ELP1 but below the Tₜ of ELP2. Aggregates of ELP1 adherent to the vessel wall are clearly identified by the bright areas of fluorescence. The bar is 100 μm.

FIG. 7. Accumulation of thermally sensitive ELP1 (T_{b}<Tₜ<Tₕ) and thermally insensitive ELP2 (Tₜ>Tₕ) in the tumor determined by window chamber (FIG. 7A), autoradiography (FIG. 7B), and radiolabel biodistribution studies (FIG. 7C). FIG. 7A, ELP accumulation in the tumor extravascular compartment was determined by separating the extravascular compartment from the vascular compartment and normalizing by the vascular intensity at t=0. FIG. 7B, Autoradiograph of ¹⁴C-ELP from 20 μm tumor sections after a 1 hour hyperthermia treatment. FIG. 7C, Tumor accumulation of ¹⁴C-ELP after a 1 hour hyperthermia treatment (n=5). All values are reported as mean±SEM and statistical significance (P-value <0.05, ANOVA) versus ELP2 (heat) and ELP1 is indicated by an *.

FIG. 8. Angular dependent dynamic light scattering data of ELP2-32,4-90 at 43° C. The change in apparent diffusion coefficient (Dₐₚₚ) with angle is indicative of a monodisperse vesicle with an outer diameter of about 272.32 nm.

### DETAILED DESCRIPTION OF THE PREFERRED EMBODIMENTS

“Affinity binding agent” as used herein may be any suitable binding partner, including antibodies in general, and tumor ligands.

“Tumor ligand” as used herein may be any compound that specifically binds to antigens in a tumor including the tumor stroma in vitro or in vivo, such as an anti-tumor antibody, antibody fragment, peptide or small molecule. Such ligands are well-known to those of skill in the art. Exemplary anti-tumor peptides include, e.g., Arg-Gly-Asp (RGD) and Asn-gly-Art (NGR), whereas anti-tumor small molecules include, but are not limited to folate or vitamin B12. See, e.g., U.S. Pat. Nos. 6,852,703; 6,676,927; 6,004,554; 5,595,721; 5,230,990; 5,177,192; 4,865,835; and 4,828,991.

“Antibody” or “antibodies” as used herein refers to all types of immunoglobulins, including IgG, IgM, IgA, IgD, and IgE. The term “immunoglobulin” includes the subtypes of these immunoglobulins, such as IgG₁, IgG₂, IgG₃, IgG₄, etc. Of these immunoglobulins, IgM and IgG are preferred, and IgG is particularly preferred. The antibodies may be of any species of origin, including (for example) mouse, rat, rabbit, horse, or human, or may be chimeric antibodies. The term “antibody” as used herein includes antibody fragments which retain the capability of binding to a target antigen, for example, Fab, F(ab′)₂, and Fv fragments, and the corresponding fragments obtained from antibodies other than IgG. Such fragments are also produced by known techniques.

“Active compound” as used herein includes, but is not limited to, therapeutic agents, and diagnostic agents such as imaging agents (radionuclides, chemotherapeutics, fluorescent compounds, etc). Non-limiting examples include, but are not limited to, 1) small molecules (e.g., organic compounds up to 700 or 2000 Da) that are chemically synthesized, including all chemotherapeutics, imaging agents and radionuclides; 2) chemically or recombinantly synthesized peptides or modified peptides; 3) peptidomimetics; and 4) protein therapeutics including antibodies and the like. See, e.g., U.S. Pat. Nos. 6,017,513; 5,965,131; and 5,958,408.

“Therapeutic agent” as used herein may be any suitable therapeutic agent including, but not limited to, radionuclides, chemotherapeutic agents, and cytototoxic agents.

“Radionuclide” as described herein may be any radionuclide suitable for delivering a therapeutic dosage of radiation to a tumor or cancer cell including, but not limited to, ²²⁷AC, ²¹¹At, ¹³¹Ba, ⁷⁷Br, ¹⁰⁹Cd, ⁵¹Cr, ⁶⁷Cu, ¹⁶⁵Dy, ¹⁵⁵Eu, ¹⁵³Gd, ¹⁹⁸Au, ¹⁶⁶Ho, ^{113m}In, ^{115m}In, ¹²³I, ¹²⁵I, ¹³¹I, ¹⁸⁹Ir, ¹⁹¹Ir, ¹⁹²Ir, ¹⁹⁴Ir, ⁵²Fe, ⁵⁵Fe, ⁵⁹Fe, ¹⁷⁷Lu, ¹⁰⁹Pd, ³²P, ²²⁶Ra, ¹⁸⁶Re, ¹⁸⁸Re, ¹⁵³Sm, ⁴⁶Sc, ⁴⁷Sc, ⁷²Se, ⁷⁵Se, ¹⁰⁵Ag, ⁸⁹Sr, ³⁵S, ¹⁷⁷Ta, ¹¹⁷mSn, ¹²¹Sn, ¹⁶⁶Yb, ¹⁶⁹Yb, ⁹⁰Y, ²¹²Bi, ¹¹⁹Sb, ¹⁹⁷Hg, ⁹⁷Ru, ¹⁰⁰Pd, ^{101m}Rh, and ²¹²Pb. Radionuclides may also be those useful for delivering a detectable dosage for imaging or diagnostic purposes, even where those compounds are not useful for therapeutic purposes.

“Chemotherapeutic agent” as used herein includes, but is not limited to, methotrexate, daunomycin, mitomycin, cisplatin, vincristine, epirubicin, fluorouracil, verapamil, cyclophosphamide, cytosine arabinoside, aminopterin, bleomycin, mitomycin C, democolcine, etoposide, mithramycin, chlorambucil, melphalan, daunorubicin, doxorubicin, tamosifen, paclitaxel, vincristin, vinblastine, camptothecin, actinomycin D, and cytarabine.

“Cytotoxic agent” as used herein includes, but is not limited to, ricin (or more particularly the ricin A chain), aclacinomycin, diphtheria toxin, monensin, Verrucarin A, Abrin, Vinca alkaloids, Tricothecenes, and Pseudomonas exotoxin A.

For the purposes of the present invention, the term “treat” or “treating” refers to any type of treatment or prevention that imparts a benefit to a subject afflicted with a disease or at risk of developing the disease, including improvement in the condition of the subject (e.g., in one or more symptoms), delay in the progression of the disease, delay the onset of symptoms or slow the progression of symptoms, etc. As such, the term “treatment” also includes prophylactic treatment of the subject to prevent the onset of symptoms. As used herein, “treatment” and “prevention” are not necessarily meant to imply cure or complete abolition of symptoms.

“Treatment effective amount” as used herein means an amount of the antibody sufficient to produce a desirable effect upon a patient inflicted with the condition being treated, including improvement in the condition of the patient (e.g., in one or more symptoms), delay in the progression of the disease, etc.

“Conjugate” as used herein refers to two or more moieties or functional groups that are covalently or non-covalently joined to one another, such that the two or more groups function together as a single structure under the conditions of the methods described herein. In one embodiment, the conjugate is a fusion protein. In another embodiment, the affinity binding agent and active compound may be covalently linked to an ELP-BC though a stable or degradable bond. As used herein, “fusion protein” refers to a protein or peptide, produced by recombinant means (i.e., expression from a nucleic acid), that is composed of a first protein or peptide covalently joined on expression to a second protein or peptide.

A “polymer that undergoes an inverse temperature transition” herein refers to a polymer that is soluble in an aqueous solution at a lower temperature, and is insoluble in an aqueous solution at a higher temperature.

For the purposes of the present invention, “transition temperature” or “Tₜ” refers to the temperature above which a polymer that undergoes an inverse temperature transition is insoluble in an aqueous system (e.g., water, physiological saline solution, blood plasma), and below which such a polymer is soluble in an aqueous system.

A “bioelastic polymer” is, in general, a polypeptide that exhibits an inverse temperature transition. Bioelastic polymers are discussed in greater detail herein. Such bioelastic polymers are typically elastin-like peptides.

“Micelle” as used herein refers to an aggregate of surfactant molecules (in this case, the ELP-block copolymers or conjugate compounds) dispersed in a liquid colloid. Micelles may be of any suitable shape, including but not limited to spherical, globular, ellipsoids, cylinders, bilayers, vesicles, etc.

While the present invention is concerned primarily with the treatment of human subjects, the invention may also be used for the treatment of animal subjects, particularly mammalian subjects such as dogs, cats, horses, cows, pigs, etc., for veterinary purposes.

Subjects in need of treatment by the methods described herein include subjects afflicted with solid tumors or cancers such as lung, colon, breast, brain, liver, prostate, spleen, muscle, ovary, pancreas, skin (including melanoma), etc.

A. Bioelastic Polymers. Bioelastic polymers are known and described in, for example, U.S. Pat. No. 5,520,672 to Urry et al. In general, bioelastic polymers are polypeptides comprising elastomeric units of bioelastic pentapeptides, tetrapeptides, and/or nonapeptides (that is, “elastin-like peptides”). Thus, in some embodiments the elastomeric unit is a pentapeptide, in other embodiments the elastomeric unit is a tetrapeptide, and in still other embodiments the elastomeric unit is a nonapeptide. Bioelastic polymers that may be used to carry out the present invention are set forth in U.S. Pat. No. 4,474,851, which describes a number of tetrapeptide, pentapeptide and hexapeptide repeating units that can be used to form a bioelastic polymer. Specific bioelastic polymers that can be used to carry out the present invention are also described in U.S. Pat. Nos. 4,132,746; 4,187,852; 4,500,700; 4,589,882; and 4,870,055. Still other examples of bioelastic polymers are set forth in U.S. Pat. No. 6,699,294 to Urry; U.S. Pat. No. 6,753,311 to Fertala and Ko; and U.S. Pat. No. 6,063,061 to Wallace.

In one embodiment, the bioelastic polymers used to carry out the present invention are polypeptides of the general formula (Val-Pro-Gly-Xaa-Gly)ₘ (SEQ ID NO:1) where Xaa is any amino acid other than proline (e.g., Ala, Leu, Phe) and m is any suitable number such as 2, 3 or 4 up to 60, 80 or 100 or more. The frequency of the various amino acid residues as the fourth amino acid can be changed, as well as the identity of Xaa. For example, the bioelastic polymers used to carry out the present invention may be polypeptides of the general formula: (Val-Pro-Gly-Xaa-Gly)ₘ (SEQ ID NO:1), where m is at least 1, 2, or 3 up to 100, 150 or 300 or more. An ELP gene is composed of a specific guest residue (Xaa) composition and can be named for simplicity as ELP2 or ELP4, as an example.

In some but not all embodiments, bioelastic polymers used to carry out the present invention may be composed of repeating elastomeric units selected from the group consisting of bioelastic pentapeptides and tetrapeptides, where the repeating units comprise amino acid residues selected from the group consisting of hydrophobic amino acid and glycine residues and where the repeating units exist in a conformation having a beta-turn of the formula:

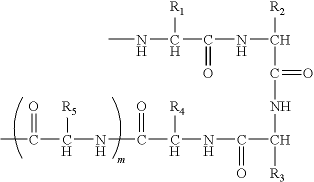

wherein R₁-R₅ represent side chains of amino acid residues 1-5 of the bioelastic polymer, and m is 0 when the repeating unit is a tetrapeptide, or 1 when the repeating unit is a pentapeptide. Nonapeptide repeating units generally consist of sequential tetra- and pentapeptides. Preferred hydrophobic amino acid residues are selected from the group consisting of alanine, valine, leucine, isoleucine, proline, phenylalanine, tryptophan, and methionine. In many cases, the first amino acid residue of the repeating unit is a residue of valine, leucine, isoleucine or phenylalanine; the second amino acid residue is a residue of proline; the third amino acid residue is a residue of glycine; and the fourth amino acid residue is glycine or a very hydrophobic residue such as tryptophan, phenylalanine or tyrosine. Particular examples include the tetrapeptide Val-Pro-Gly-Gly (SEQ ID NO:2), the tetrapeptide Gly-Gly-Val-Pro (SEQ ID NO:3), the tetrapeptide Gly-Gly-Phe-Pro (SEQ ID NO:4), the tetrapeptide Gly-Gly-Ala-Pro (SEQ ID NO:5), the pentapeptide Val-Pro-Gly-Val-Gly (SEQ ID NO:6), the pentapeptide Gly-Val-Gly-Val-Pro (SEQ ID NO:7), the pentapeptide Gly-Lys-Gly-Val-Pro (SEQ ID NO:8), the pentapeptide Gly-Val-Gly-Phe-Pro (SEQ ID NO:9), the pentapeptide Gly-Phe-Gly-Phe-Pro (SEQ ID NO:10), the pentapeptide Gly-Glu-Gly-Val-Pro (SEQ ID NO:11), the pentapeptide Gly-Phe-Gly-Val-Pro (SEQ ID NO:12), and the pentapeptide Gly-Val-Gly-Ile-Pro (SEQ ID NO:13). See, e.g., U.S. Pat. No. 6,699,294 to Urry.

B. Copolymers and Conjugates. In general, the block copolymer comprises, consists of, or consists essentially of (i) a first elastin-like polypeptide (ELP) having a first Tₜ and (ii) a second elastin-like polypeptide having a second Tₜ greater than said first Tₜ. In general, the first Tₜ is at least 10° C. or 20° C., but not more than about 50° C. or 60° C. In general, the second Tₜ is at least 30° C. or 40° C., but not more than 90° C. or 100° C.

In one embodiment of the block copolymer, the first ELP is positioned at the N-terminus and the second ELP is positioned at the C-terminus thereof; in another embodiment of the block copolymer the first ELP is positioned at the C-terminus and the second ELP is positioned at the N-terminus thereof.

In one embodiment of the conjugate, the compound of interest (e.g., the diagnostic or therapeutic agent) is coupled to the C-terminus and the affinity binding agent (e.g., the antibody) is coupled to the N-terminus of the block copolymer. In another embodiment of the conjugate, the compound of interest is coupled to the N-terminus and the affinity binding agent is coupled to the C-terminus of the block copolymer. The compound of interest and affinity binding agent may also be attached at various frequencies, locations and ratios throughout the entire ELP protein.

Coupling of conjugates can be carried out by any suitable means, such as by recombinant means where elastin is joined to a protein or peptide such as GFP; by expression of a fusion protein; by chemical means where the compound to be coupled to the ELP by a chemical reaction or by enzymatic coupling to provide a covalent linkage between the active compound and the ELP; by noncovalent means such as chelation or hydrophobic interactions; etc. Ligands can be attached to the ELP with any of these coupling schemes above. The ligand can be either a protein or peptide, such as a single-chain antibody fragment or peptide (e.g., RGD or NGR); or a chemical entity such as folate. Moreover, the linkage between the compound of interest and affinity binding agent may be stable or labile.

In some embodiments, the block copolymer consists of from 1, 2, 3 or 4 blocks (with each “block” consisting of a single first-elastin-like polypeptide with a specific gene sequence, coupled to a single second elastin-like polypeptide). In a particular embodiment, the block copolymer consists of 2 blocks.

Block copolymers and conjugates thereof can be formed into micelles in accordance with known techniques, including but not limited to those described in U.S. Pat. Nos. 6,951,655; 6,835,718; 6,780,324; and 5,858,398.

In some embodiments of the compositions and methods of the invention, the block copolymer or conjugate compound is provided in the form of a particle such as a vesicle such as a liposome or micelle (e.g., at normal physiological temperature). A ligand (such as an antibody) that specifically binds to a binding partner in the tumor can be coupled to the particle in accordance with known techniques to facilitate targeting of the micelle to a tumor.

In some embodiments the block copolymer is formed into micelles (e.g., micelles which retain their micellar structure at normal physiological temperature) and release active compounds incorporated therein in response to an external or internal stimulus such as heating. The release of active compounds may be caused by changes in the corona's properties, core segment disassembly or degradation of ELP-BC constituents. This embodiment may be combined with that in which a tumor ligand is incorporated into the micelle or vesicle, as described above.

In some embodiments the ELP-BC can be designed to target solid tumors by presenting tumor ligands in the corona of the micelle and exploiting polyvalent binding only in the tumor. In some embodiments this is accomplished by intravenously administering the ELP-BCs and using and external or internal stimulus such as heating the tumor with externally focused hyperthermia to temperatures >40° C. The stimulus will only be present in the tumor therefore selectively forming polyvalent particles only in the tumor. The greater avidity of polyvalent interactions (Mammen, et al. (1998) Angewandte Chemie-Intl. Ed. 37(20):2755-2794) will localize more anticancer drugs in the tumor due to recognition of tumor-specific or tumor-associated antigens and limit systemic exposure and toxicity. This approach overcomes the current problems with affinity targeted drug delivery such as non-specific binding to normal tissues, poor penetration and lack of an ubiquitous ligand for the majority of cancer patients.

In some embodiments, the block copolymers and conjugates are formed into micelles that have a crosslinked core. For example, the block copolymers and conjugates can be synthesized to form micelles at any reasonable temperature and contain cysteine or suitable other residues in the hydrophobic segment. The micelle formation will be induced with temperature under reducing conditions, and then the reducing environment will be removed to create disulfide bonds within the core of the micelle. This procedure will result in a micellular geometry that does not require an elevated temperature because the micelle structure will be stabilized by the disulfide bonds within the micelle's core. The properties of the micelle such as size and aggregation number will be controlled by varying the ELP-BC segment length. In this example, drugs may also be incorporated with a linkage terminated with a free thiol to also be contained in the core of the micelle during self-assembly and crosslinking.

In some embodiments, the block copolymers or conjugates are formed into vesicles. For example, ELP block copolymers can form vesicles when the hydrophilic and hydrophobic block's gene identity and molecular weight are properly chosen. These vesicles can be used to passively deliver active compounds similar to liposomes and polymersomes or can be designed to rupture with some external or internal stimulus. These vesicles can also be crosslinked to form more stable vesicular structures. This approach may be combined with a tumor ligand presented on the surface of the vesicle.

In some embodiments, liposomes can be formed or later modified to contain ELP segments that can be triggered with an internal or external stimulus to enhance drug release. These ELPs may also be contained within the aqueous phase of the liposome to burst the liposome upon an internal or external stimulus. The ELPs may also enhance tumor accumulation by protecting the liposome from the reticuloendothelial system or by selectively inducing the phase transition in the tumor.

In some embodiments, the block copolymers are synthesized so that the two blocks are linked to each other by a disulfide linkage. In a reducing environment these disulfides are converted to thiols, thereby breaking the bond that holds the two segments together, resulting in disassembly of the ELP-BC self-assembled structure (e.g., micelles or vesicles). These disulfide-linked block copolymers may contain blocks composed of an ELP or a synthetic molecules such as poly-ethylene glycol and poly-ethylene oxide.

In some embodiments block copolymers of the invention, including, single-segment ELPs or multi-block ELPs, can contain side-chains that can be oxidized. For example, in single-segment ELPs, the ELP can have a Tₜ below body temperature so that upon local injection it will undergo its phase transition and be localized in the tumor or other pathologic site. Oxidation will increase the Tₜ above body temperature, so that the ELP will become soluble leading to the release of encapsulated drug or diffusion of an ELP-drug conjugate. In the latter case, the ELP-drug conjugate is labile, resulting in release of the drug by pH or other stimulus. In the case of diblock ELPs, the inner core segment will be an oxidizable segment. The diblock ELP can, in some embodiments, be such that it will be in micelle or vesicle form at body temperature with active compounds that are encapsulated on conjugated to the inner segment. In vivo oxidation of the core block will cause disassembly of the self-assembled structure, resulting in release of the active compound. Again active compound can be physically encapsulated, or conjugated or a combination of the two.

In some embodiments, the active agent is encapsulated in the hydrophobic or aqueous phase of a self assembled ELP structure, rather than coupling it to the block copolymer to form a conjugate.

In some embodiments the active agent (or a plurality of active agents) is/are both coupled to the block copolymer to form a conjugate, and encapsulated within the aqueous or hydrophobic phase of a micelle or vesicle formed from such conjugates (e.g., to enhance loading efficiency).

Preferably, the attachment or encapsulation of the drug results in improved solubility and plasma half-life of the drug.

C. Formulations and Administration. Administration of the conjugate to a subject can be carried out by any suitable means, such as subcutaneous injection, intraperitoneal injection, intraveneous injection, intramuscular injection, oral administration, inhalation administration, transdermal administration, etc. Preferred administration techniques are typically “systemic” in that a particular region of interest is not specifically targeted.

The selected region may be any suitable target or portion of the subject's body, such as a limb, organ, or other tissue or tissue portion. The selected region may be comprised of hyperproliferative tissue, which may be malignant or non-malignant, such as a solid tumor. Examples of tumors, cancers and neoplastic tissue that can be treated by the present invention include, but are not limited to, malignant disorders such as breast cancers; osteosarcomas; angiosarcomas; fibrosarcomas and other sarcomas; leukemias; lymphomas; sinus tumors; ovarian, uretal, bladder, prostate and other genitourinary cancers; colon esophageal and stomach cancers and other gastrointestinal cancers; lung cancers; myelomas; pancreatic cancers; liver cancers; kidney cancers; endocrine cancers; skin cancers; and brain or central and peripheral nervous (CNS) system tumors, malignant or benign, including gliomas and neuroblastomas. Examples of premalignant and normeoplastic hyperproliferative disorders include, but are not limited to, myelodysplastic disorders; cervical carcinoma-in-situ; familial intestinal polyposes such as Gardner syndrome; oral leukoplakias; histiocytoses; keloids; hemangiomas; etc.

Heating of the selected region can be carried out by any means, such as by application of a heat source, e.g., a heat pad, a hot water bath, infrared heating lamps, etc., or by a heating means such as directing microwave, ultrasound or other radiofrequency energy at the selected region.

Any suitable compound for which targeted delivery is desired can be administered by this means, including imaging agents (or contrast agents) and therapeutic agents. In a preferred embodiment, the therapeutic agent is a radionuclide. Any radionuclide, whether it be for therapeutic or imaging purposes, can be employed, including but not limited to, ¹³¹I, ⁹⁰Y, ²¹²At, ²¹²Bi, ⁶⁷Cu, ¹⁸⁶Re, ¹⁸⁶Re, and ²¹²Pb.

The conjugates (or “active compounds”) described above can be formulated for administration in a single pharmaceutical carrier or in separate pharmaceutical carriers for the treatment of a variety of conditions. In the manufacture of a pharmaceutical formulation according to the invention, the active compounds including the physiologically acceptable salts thereof, or the acid derivatives of either thereof are typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.5% to 95% by weight of the active compound. One or more active compounds may be incorporated in the formulations of the invention, which may be prepared by any of the well known techniques of pharmacy consisting essentially of admixing the components, optionally including one or more accessory ingredients.

The formulations of the invention include those suitable for oral, rectal, topical, buccal (e.g., sub-lingual), parenteral (e.g., subcutaneous, intramuscular, intradermal, or intravenous) and transdermal administration, although the most suitable route in any given case will depend on the nature and severity of the condition being treated and on the nature of the particular active compound which is being used.

Formulations suitable for oral administration may be presented in discrete units, such as capsules, sachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder. Formulations of the present invention suitable for parenteral administration conveniently comprise sterile aqueous preparations of the active compound, which preparations are preferably isotonic with the blood of the intended recipient. These preparations may be administered by means of subcutaneous, intravenous, intramuscular, or intradermal injection. Such preparations may conveniently be prepared by admixing the compound with water or a glycine buffer and rendering the resulting solution sterile and isotonic with the blood.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Tyle (1986) Pharmaceutical Res. 3(6):318-26) and typically take the form of an optionally buffered aqueous solution of the active compound. Suitable formulations comprise citrate or bis/tris buffer (pH 6) or ethanol/water and contain from 0.1 to 0.2 M active ingredient. The therapeutically effective dosage of any one active agent, the use of which is in the scope of present invention, will vary somewhat from compound to compound, patient to patient, and will depend upon factors such as the condition of the patient and the route of delivery. Such dosages can be determined in accordance with routine pharmacological procedures known to those skilled in the art, particularly in light of the disclosure provided herein. In one example, the dosage is from 1 to 10 micrograms of active compound per Kilogram subject body weight.

In another example, where the therapeutic agent is ¹³¹I, the dosage to the patient is typically from 10 mCi to 100, 300 or even 500 mCi. Stated otherwise, where the therapeutic agent is ¹³¹I, the dosage to the patient is typically from 5,000 Rads to 100,000 Rads (preferably at least 13,000 Rads, or even at least 50,000 Rads). Doses for other radionuclides are typically selected so that the tumoricidal dosage is equivalent to the foregoing range for ¹³¹I.

D. Internal and External Stimuli. Internal and external stimuli may be carried out by any suitable means, such as with hyperthermia, hypothermia, electromagnetic radiation (light), magnetic fields, ultrasound, pH, hypoxia, redox potential, thiol concentration, phosphorylation, cross-linking with enzymes such as tissue transglutaminase, degradation with enzymes such as matrix metalloproteases and optothermally. The internal stimuli are a property of the site that may exist naturally or be induced to exist with additional manipulation.

The present invention is explained in greater detail in the following non-limiting examples.

### Example 1

### Construction of ELP-BCs

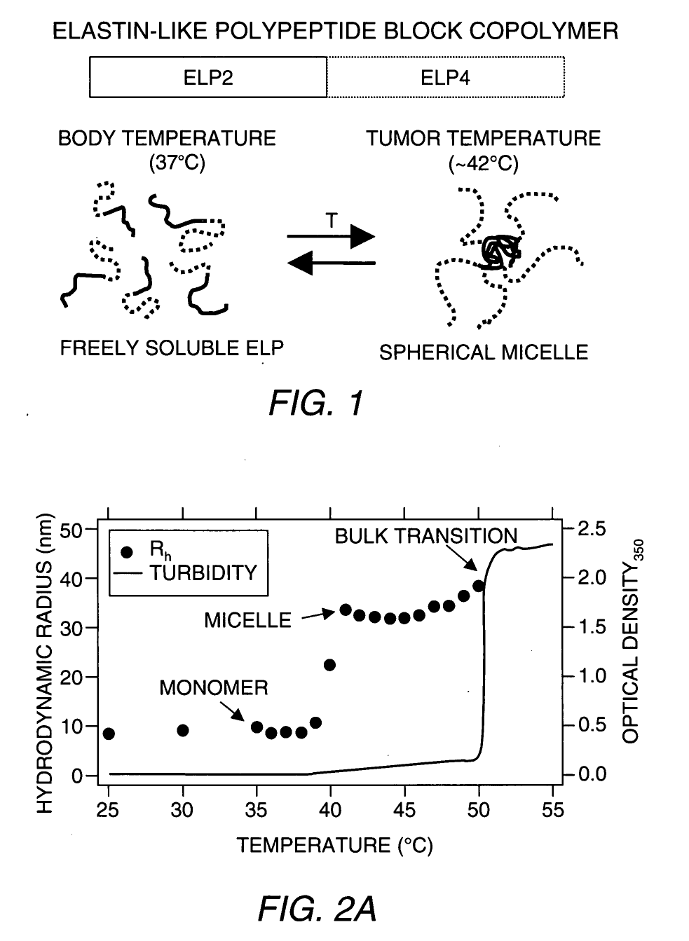

ELP-BCs were constructed as shown in FIG. 1 by seamlessly fusing two ELP genes together with different guest residue (i.e., Xaa) compositions such that the N-terminal gene had high Tₜ, termed ELP2 (Tₜ>90° C.), and the other gene at the C-terminus, called ELP4, had a lower Tₜ (Tₜ˜40° C.). The ELP-BC was highly soluble at temperatures below the Tₜ of both ELP blocks. At intermediate temperatures between the Tₜ of ELP4 and ELP2, the ELP-BC self-assembled into spherical micelles when the size and ratio of the blocks were selected correctly. The notation for the ELP-BCs consisted of the ELP gene followed by the number of pentapeptides. For example, ELP2-96,4-60 was an ELP-BC with 96 pentapeptides of an ELP2 gene at the N-terminus followed by 60 pentapeptides of ELP4 at the C-terminus. The ELP2-96,4-60 had an approximate hydrophilic (ELP2) to hydrophobic (ELP4) ratio of 3:2.

### Example 2

### ELP Drug Delivery Schemes and Applications

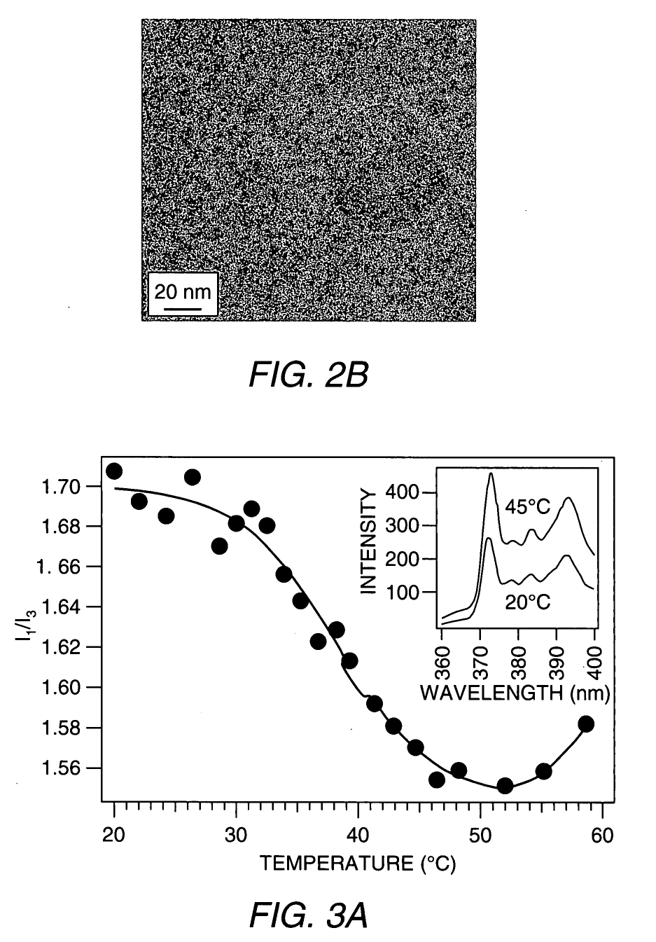

ELP-BC Micelle. As shown in FIG. 2, the ELP-BCs formed spherical micelles when heated to the appropriate temperature. In this example, the drug can be covalently linked or complexed with the core of the micelle. The corona of the micelle may or may not contain ligands to actively target a body site.

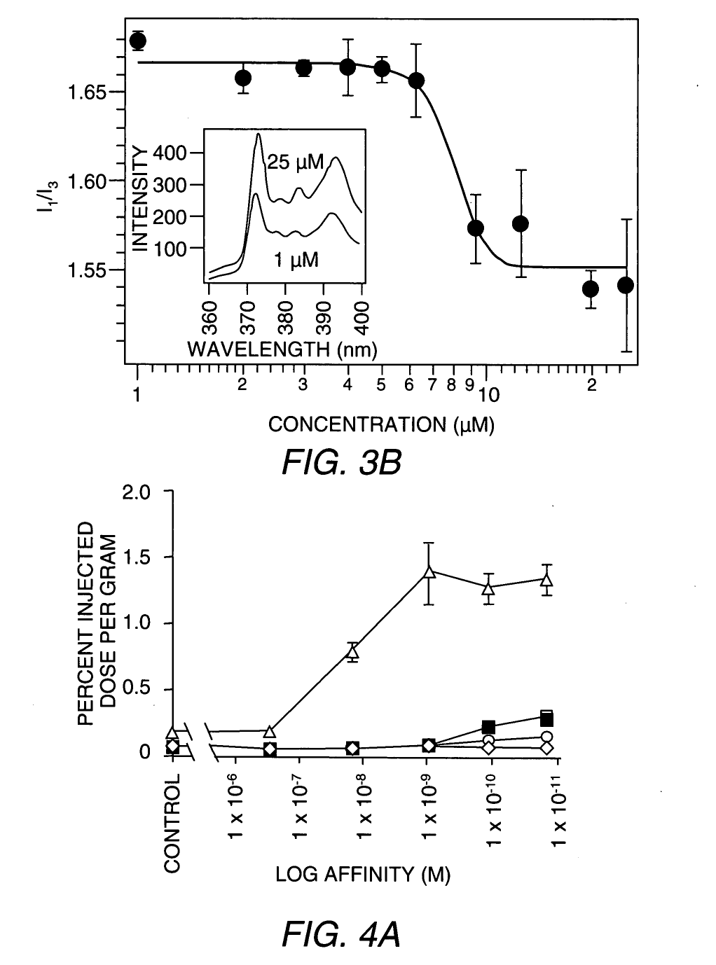

ELP-BC Stimuli-Induced Drug Release. The ELP-BC micelle can also be used to release drugs based on an internal or external stimuli such as heat as shown in FIG. 3A. The ratio of pyrene's I₁ and I₃ vibrational peaks was proportional to the polarity of pyrene's environment. As the ELP-BC formed a micelle the ratio decreased (T=20 to 48° C.) as pyrene partitioned into the hydrophobic core. This also demonstrates how drugs can be encapsulated into the core of the micelle. Upon the second phase transition at 50° C. the ratio increased, indicative of release of pyrene from the core of the micelle. These data also demonstrate the release of drug due to a specific stimulus, in this case heat.

The critical micelle concentration (CMC) was determined by plotting the minimum of the I₁/I₃ ratio from 35° C.-45° C. (see FIG. 3A) as a function of ELP-BC concentration (FIG. 3B). The inflection point of a sigmoid fit to data in this temperature range was defined as the CMC. The CMC was determined for six ELP-BCs capable of forming micelle. This ratio remained constant at low concentrations of ELP2-64,4-90, but decreased to a lower value as the concentration was increased. These data demonstrate that ELP2-64,4-90 micelles formed at a low concentration of 8.1 μM. Furthermore, all six of the micelle-forming ELP-BCs have a CMC<10 μM, indicating that ELP-BC micelles are quite stable structures.

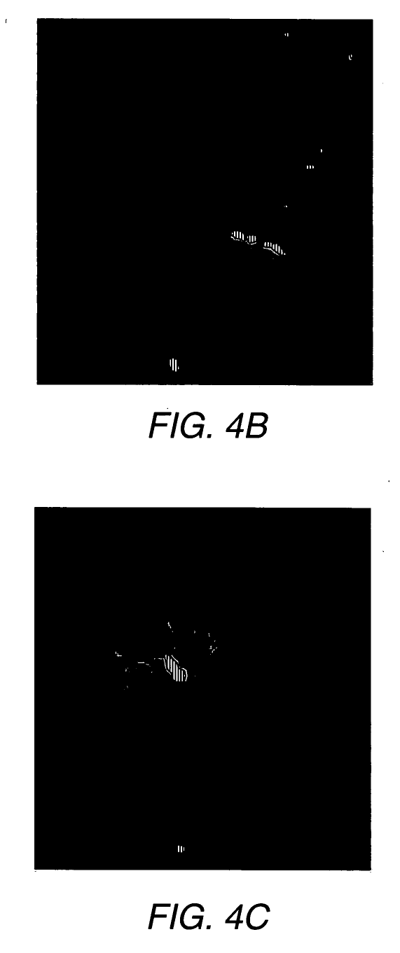

ELP-BC Micelle Affinity Modulation. Affinity modulating particles that specifically target solid tumors is a useful paradigm for targeted delivery. The efficacy of an affinity-targeted drug delivery system depends on at least these three components: 1) the accumulation in the tumor, 2) the accumulation in normal tissue, and 3) the spatial distribution within the tumor compartment. Tumoral accumulation appears to depend on the affinity of the targeting molecule. It has been found that affinities greater than 10⁻⁷ M (K_{D}) are necessary for accumulation in the tumor to be significantly greater than a control single-chain antibody fragment (shown in FIG. 4A). The accumulation increased with affinity but became constant over a range of 10⁻⁹ M to 10⁻¹¹ M (Adams, et al. (2001) Cancer Res. 61(12):4750-4755). Moreover, the cytotoxicity of targeted therapies is positively correlated with the affinity (McCall, et al. (2001) J. Immunol. 166(10):6112-6117). These data indicate that there is both a threshold affinity (˜10⁻⁷ M) to enhance tumor accumulation and a maximum affinity (˜10⁻⁹ M) above which tumor accumulation is not improved.

Along with the improved accumulation observed with higher affinities, there is also a decrease in tumor penetration of the targeting molecule known as the binding site barrier. This decreased penetration is due to the binding of a targeting molecule (i.e., ligand) to its antigen (i.e., receptor) on the surface of the cancer cell as it extravasates from its vascular source (Weinstein, et al. (1987) Ann. NY Acad. Sci. 507:199-210; Fujimori, et al. (1989) Cancer Res. 49(20):5656-5663). This binding limits the speed at which the targeting molecule can percolate through the tumor interstitium and causes very high affinity targeting molecules to be confined within only 2-3 cell diameters of the vasculature as shown in FIG. 4C (Adams, et al. (2001) Cancer Res. 61(12):4750-4755). In this scenario, only tumor cells near the vessel will be exposed to the therapeutic agent, potentially decreasing the efficacy of treatment.

The dichotomous nature of high affinity targeting makes one wonder what affinity yields the greatest therapeutic benefit? A high affinity targeted molecule (>10⁻⁹ M) would have near maximal tumor accumulation, but its tumor penetration is limited. Tumor penetration is increased with low affinity antibodies (<10⁻⁹ M), while simultaneously, sacrificing accumulation. The greatest therapeutic benefit may be obtained with an affinity targeting molecule that initially has a high affinity for its tumor receptor to result in near maximal accumulation, followed by a decrease in affinity to facilitate penetration.

Modulated affinity targeting can be realized through exploiting the principles of polyvalency. Polyvalent molecules tend to have a higher “avidity” (defined as the effective affinity of a polyvalent interaction) than the affinity of an equivalent monovalent association. As disclosed herein, it has been shown that ELP-BCs form polyvalent micelles in response to an increase in solution temperature. By decorating the corona of the micelle with ligands for tumor receptors, the avidity of the ELP-BC particles can be modulated simply through a change in solution temperature.

The application of this technology would involve intravenous administration of free ELP-BCs conjugated to a ligand that binds a tumor receptor. The tumor is heated with externally focused hyperthermia. In normal systemic circulation, the ELP-BC will be freely soluble in its monovalent form and therefore have a low affinity. Upon entering the tumor vasculature the solution temperature is increased and the ELP-BC forms polyvalent micelles. The higher avidity micelles will accumulate in the tumor according to their avidity and hydrodynamic radius. After the hyperthermia treatment is completed (1-2 hours), the ELP-BC micelles will potentially dissociate into their monovalent components to facilitate diffusion and penetration into the tumor.

In addition to combining high affinity accumulation with low affinity penetration, this strategy will also limit systemic accumulation. If the target tumor receptor is tumor-associated, not tumor-specific, then the receptor will be presented on many cells throughout the body. The monovalent ELP biopolymer would be designed to have an affinity ≦10⁻⁷ M to reduce accumulation in normal tissue. The ELP-BC micelle's avidity would increase between 10³- and 10⁸-fold in the tumor from its monovalent form in normal tissues (Mammen, et al. (1998) Angewandte Chemie-Intl. Ed. 37(20):2755-2794). The selectively higher avidity within the tumor allows for the use tumor-associated antigens that are expressed on many clinically relevant cancers thus making this strategy more widely applicable than other affinity targeting techniques. It is contemplated that the affinity modulated ELP-BC particles will exhibit: 1) substantial levels of tumor accumulation due to their high affinity selectively in the tumor, 2) limited systemic exposure because of the low affinity of the monovalent interaction with normal tissues and 3) better penetration into tumors upon the completion of the hyperthermia treatment.

Relevant in vivo ligands can also be attached to the N-terminus of the ELP block in the micelle corona. The ligands can be specific to endothelial cell receptors in addition to tumor cell receptors. Examples include, e.g., the RGD peptide and folate as an endothelial and tumor cell-specific ligand, respectively. The RGD peptide has been found to bind to endothelial cells of solid tumors (Arap, et al. (1998) Science 279(5349):377-380). Folate is a vitamin required for several cellular metabolic pathways and is internalized through receptor-mediated endocytosis after binding to the folate receptor (FR). One hallmark of cancer is the rapid proliferation of cancer cells (Hanahan & Weinberg (2000) Cell 100(1):57-70) and because folate is essential for the biosynthesis of nucleotide bases, the FR is frequently over-expressed, by up to 2 orders of magnitude, on the surface of cancer cells. The FR has been found to be over-expressed in many malignancies including cancer of the ovary, brain, kidney and the lung. This pattern of over-expression make the FR a useful tumor-associated antigen Lu & Low (2003) J. Contr. Rel. 91(1-2):17-29). Furthermore, the ELP-BC can be functionalized with moieties that that will change the physicochemical properties of the micelle surface after self-assembly. Examples include, e.g., arginine which would make a net positive charge on the surface of the micelle thereby facilitating internalization of ELP micelles over ELP unimers.

Type of therapeutics conducive to this novel delivery method include, but are not limited to, chemotherapeutics or radionuclides. It is believed that a large antivascular effect can be realized by combining high LET radiation therapy (e.g., ²¹¹At) with ELP-BCs that target endothelial specific receptors. However, a more profound anti-tumor effect may be gained by combining chemotherapy with tumor cell-specific ligands. The therapeutic agents can be conjugated to a C-terminal cysteine of the ELP using conventional maleimido chemistry. Doxorubicin or other chemotherapy agents can be linked to the ELP-BC through a conventional hydrazone bond that degrades in the low pH environment of the lysosome once internalized by cells in order to facilitate site-specific toxicity. The ²¹¹At or other radionuclides could be linked to the ELP through a stable linkage. Since the drug is attached to the C-terminus, it should be buried in the core of the micelle upon self assembly and not interfere with binding of the corona to specific targets.

### Example 3

### Analysis of ELP-BC Micelle Affinity Modulation

In preliminary studies, experiments were conducted to demonstrate the feasibility of the affinity modulation approach. In order for ELP-BCs to effectively modulate affinity for tumor-associated targets, three characteristics were sought; 1) ELP-BCs form spherical micelles in response to an increase in temperature, 2) the micelle formation temperature and size can be controlled and rationally designed a priori, and 3) the phase transition of the ELP can be triggered in vivo. The following describes the results which demonstrate that these requirements were effectively met.

ELP-BCs Form Spherical Micelles. Ten different ELP-BCs were constructed with various molecular weights and hydrophilic to hydrophobic ratios (i.e., ratio of ELP2 to ELP4), of which six out of the 10 formed spherical micelles when heated to intermediate temperatures between the Tₜ of both ELP blocks. It was empirically determined that the ratio of hydrophilic to hydrophobic blocks should be between 1:2 and 2:1 in order for the ELP-BCs to properly self-assemble into micelles. The temperature-dependent self-assembly of ELP-BCs determined by UV-vis spectrophotometry and dynamic light scattering (DLS) is shown in FIG. 2A. The ELP was highly soluble as a monomer at low temperatures but formed a micelle at 40° C. due to the hydrophobic transition of the low temperature block. This micelle persisted up to 50° C., where the coronal block underwent its transition and a bulk aggregate was formed. The formation of spherical micelles was confirmed by vitrifying an ELP-BC from a temperature that induced micelle formation and imaging the samples with cryo-TEM as shown in FIG. 2B. The micelle size determined by DLS and cryo-TEM were nearly identical. Angular dependent DLS studies indicated that the ELP-BC formed exquisitely monodisperse micelles since no angular dependence could be detected. These studies indicate that ELP-BCs form monodisperse spherical micelles in response to an increase in solution temperature.

Micelle Formation Temperature and Size is Rationally Controlled. The micelle formation temperature was determined by the molecular of the low temperature block. It has been shown that the T, of the ELP is inversely proportional to its molecular weight (Meyer & Chilkoti (2004) Biomacromolecules 5(3):846-851), therefore increasing the molecular weight of the low temperature block reduced the micelle formation temperature as shown in Table 1. The micelle formation temperature occurred ˜4° C. higher for the ELP-BC than the parent ELP4 possibly due to the influence of the ELP2 segment on ELP4's thermal properties. The size of the micelle was influenced by the molecular weight and the ratio of hydrophilic to hydrophobic blocks (see Table 1). For example, as the hydrophobic block fraction became smaller, the Rₕ decreased for a consistent molecular weight (see molecular weight ≅74 kDa). Furthermore, as the total molecular weight was raised and the ratio of blocks was held constant (e.g., 1:1) the average size of the micelle also increased. The R_{g}/Rₕ values were close to the predicted value of 0.775 for a solid sphere which would be expected for a micelle. The high coordination numbers indicate that the ELP-BCs do self-assemble into structures capable of presenting polyvalent ligands. These studies indicate that the micelle formation temperature and physical properties such as size can be controlled by rationally selecting the molecular weight and block ratio.

TABLE 1

| | | | Micelle | ELP4 | | | | |
|---|---|---|---|---|---|---|---|---|
| | MW | | temp | Tₜ | | | | Coordination |
| ELP | (kDa) | Ratio | (° C.) | (° C.) | Rₕ (nm) | R_{g} (nm) | R_{g}/Rₕ | number |
| ELP2-64,4-120 | 74.1 | 1:2 | 33 | 28.7 | 39.9 | 30.0 | 0.753 | 116 |
| ELP2-96,4-90 | 73.9 | 1:1 | 36 | 31.0 | 36.6 | 26.5 | 0.724 | 133 |
| ELP2-128,4-60 | 75.1 | 2:1 | 42 | 35.5 | 34.3 | 24.0 | 0.701 | 56 |
| ELP2-64,4-90 | 62.8 | 2:3 | 36 | 31.0 | 30.0 | 19.0 | 0.633 | 110 |
| ELP2-96,4-60 | 63.1 | 3:2 | 42 | 35.5 | 32.0 | 22.0 | 0.687 | 94 |
| ELP2-64,4-60 | 49.4 | 1:1 | 42 | 35.5 | 29.1 | 20.7 | 0.712 | 57 |

The ratio is expressed an approximate length ratio of hydrophilic to hydrophobic blocks.
The temperature the micelle forms is defined as the micelle temperature.
ELP4 Tₜ is defined as the maximum in dOD/dT from an upward thermal ramp (1° C./minute) in PBS at 25 μM.
The Rₕ was determined from intercept of q² dependence from DLS experiments and the R_{g} was determined from static light scattering experiments.
The coordination number was calculated by dividing the apparent molecular weight from static light scattering experiments by the monomer's molecular weight.

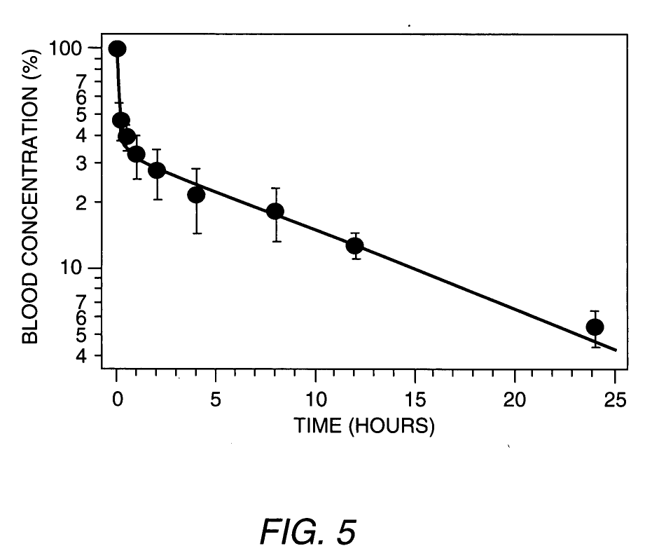

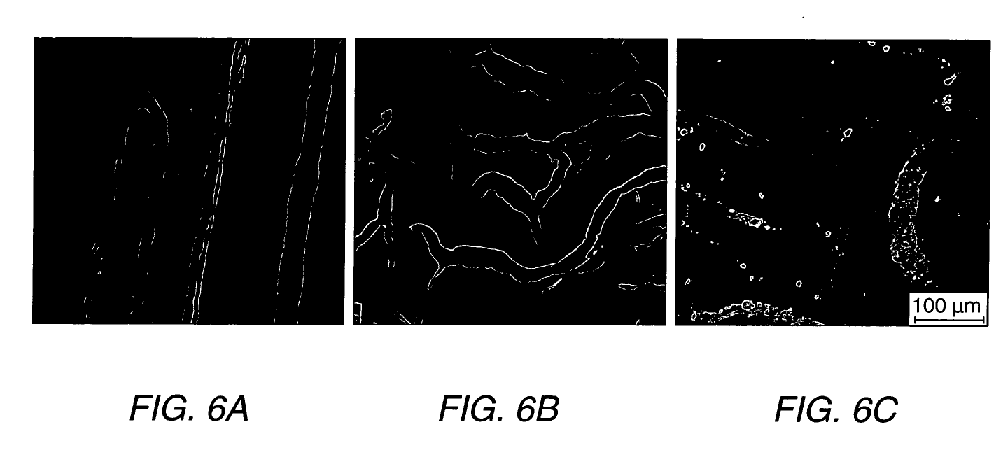

ELP Phase Transition can be Triggered in vivo. Pseudorandom copolymer ELPs or “normal” ELPs that consist of a single gene as an active macromolecular drug carrier are also contemplated. In the same manner as with the ELP-BCs, the phase transition of the ELP is induced only in the tumor by heating the tumor with externally focused hyperthermia and tuning the Tₜ of the ELP to be greater than body temperature (T_{b}=37° C.), but less than the hyperthermic tumor temperature (Tₕ=42° C.). The ELP will be highly soluble in normal circulation; however, upon entering the hyperthermic tumor, the ELP will undergo its phase transition (T_{b}<Tₜ<Tₕ) and accumulate in the tumor. To investigate the influence of the phase transition in vivo, intravital microscopy, radiolabel tumor accumulation, and radiolabel pharmacokinetic studies were performed. The ELP exhibited characteristic distribution and elimination response for macromolecules after intravenous administration (FIG. 5). Novel tools were created using the dorsal skin fold window chamber model and laser scanning confocal microscopy to quantify the relative concentration in the vascular and extravascular compartments of the tumor. Representative images of normal and tumor vasculature are shown in FIG. 6. The normal vasculature (FIG. 6A) exhibited a classic Krogh's cylinder geometry. In contrast, the tumor vasculature (FIG. 6B) was chaotic with uneven and dilated vessels that appeared to initiate angiogenic spouting. To determine if the ELP phase transition could be induced in vivo, the tumor was heated to 42° C. (Tₕ) and an ELP with a T₁<42° C. (ELP1) and an ELP with a Tₜ>42° C. (ELP2) were co-injected. The clearly visibly bright aggregates of ELP1 adherent to the tumor blood vessel wall shown in FIG. 6C indicate that the phase transition can be induced in vivo. The control ELP2 with a Tₜ>42° C. did not exhibit any punctuate fluorescence (FIG. 6C).

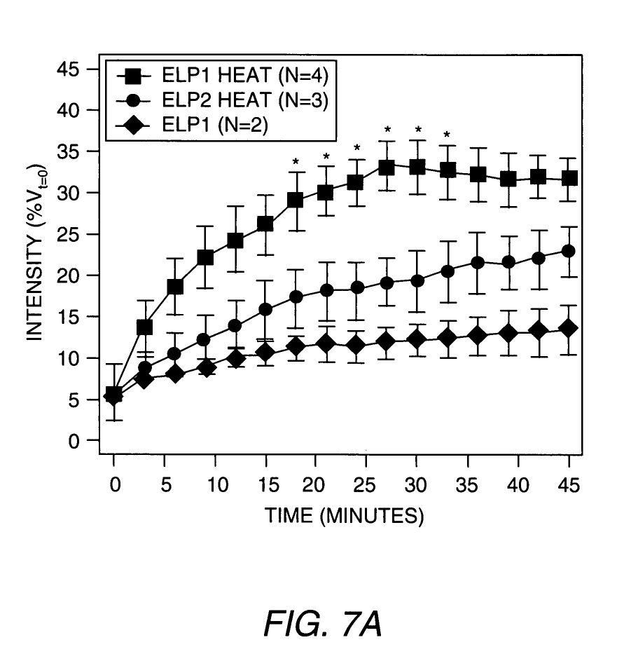

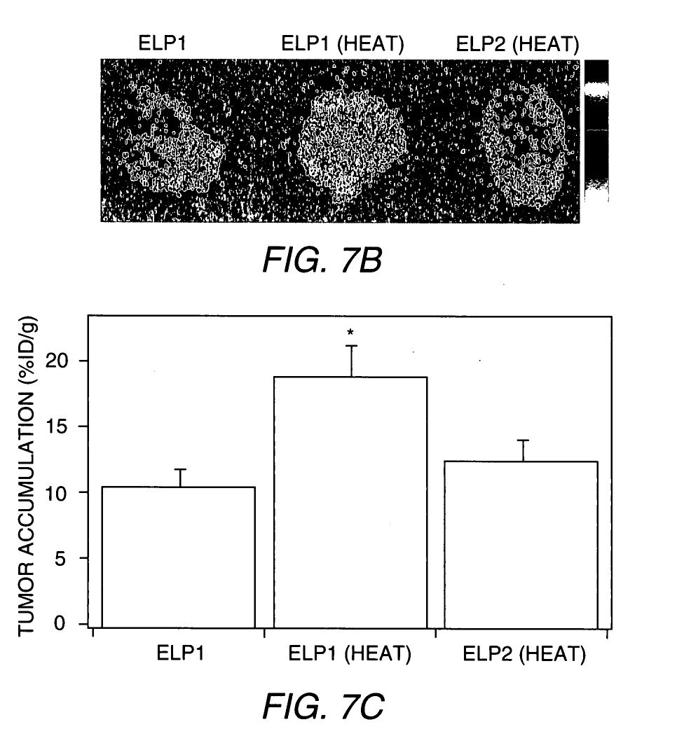

To examine the exposure of cancer cells to the ELP, the fluorescence intensity was calculated in the extravascular compartment normalized by the initial vascular intensity through a series of images as shown in FIG. 7A. The thermally sensitive ELP1 without the application of hyperthermia had modest extravascular accumulation. The nonspecific effect of hyperthermia on permeability and perfusion was illustrated by the increased accumulation of the thermally insensitive ELP2 with hyperthermia. Moreover, when a thermally sensitive ELP1 was administered in combination with hyperthermia, the extravascular accumulation was increased to it highest levels. This increased accumulation of a thermally sensitive ELP in combination with externally applied hyperthermia was mirrored by the autoradiography and radiolabel tumor accumulation studies as shown in FIG. 7B and FIG. 7C, respectively. These studies indicate that the phase transition of the ELP can be tuned to occur in the narrow temperature range between the body and hyperthermic tumor temperature (T_{b}<Tₜ<Tₕ) to result in increased tumor accumulation. These results indicate that ELP-BCs can also be tuned to selectively undergo their phase transition within the tumor in vivo.

### Example 4

### Additional Modifications

A number of ELP-BCs were produced that formed spherical micelles at the appropriate temperature (˜40° C.) for use in externally targeted hyperthermia to trigger polyvalent interactions only in a solid tumor. In addition to this strategy, the ELP-BC, therapeutic agent, ligand and stimulus may be modified to further improve efficacy. A number of these modifications are listed below.

1) Despite a very high coordination number (>100), the ELP-BCs may not present enough ligands on the surface of the micelle to take part in polyvalent interactions. To remedy this, many ligands are attached on the N-terminal (hydrophilic) segment of the ELP-BC to increase the probability of being solvent exposed once in a micelle configuration.

2) The therapeutic agents that can be delivered include chemotherapeutics, radionuclides and biological agents such as proteins.

3) The therapeutic agents can be attached covalently or chelated to the ELP. The linkage between the ELP-BC and therapeutic can be physiologically stable or degradable depending on a specific stimulus such as pH or native enzymes.

4) The ligand can target tumor-associated or tumor-specific receptors. The receptor can be on the tumor endothelium, within the tumor stroma or on the cancer cell itself. The ligand can be a short peptide (e.g., RGD), protein (e.g., antibody fragment) or a chemically synthesized entity (e.g., folate).

5) The ligand can be covalently attached to the ELP at any location or incorporated into the protein itself. The linkage between the ELP-BC and ligand can be physiologically stable or degradable depending on a specific stimulus such as pH or native enzymes.

6) The ligand can be selected to facilitate cellular internalization or simply remain attached to the cell surface. If internalized, the ELP-BC can be targeted to the lysosomal pathway or not.

7) The stimulus to create a polyvalent particle can be external or internal. Examples of an external stimulus include, but are not limited to, heat, electromagnetic radiation (light), magnetic field and ultrasound. Examples of an internal stimulus would be low pH, hypoxia, specific enzymes, thiol concentration, phosphorylation, optothermally and redox potential.

8) In addition to the ELP-BCs disclosed herein, other polymers may be able to self-assemble into polyvalent particles in response to a stimulus. For example poly-NIPAAM and PEG block copolymers may exhibit similar behavior.

### Example 5

### Efficacy of Micelles, Affinity and Thermal Targeting

Many interactions in biological systems are polyvalent in nature such as the association between cells, recognition of antigens by the immune system and binding of transcription factors to DNA (Mammen, et al. (1998) Angewandte Chemie-Intl. Ed. 37(20):2755-2794). Moreover, the monovalent equivalent of these interactions often does not have a sufficient affinity, thus requiring polyvalent interactions for normal physiologic homeostasis. Polyvalent binding exhibits enhanced avidity compared to the same monovalent interaction most likely through a diminished rate of dissociation (k_{off}). The thermodynamic cost of the first binding event (kₒₙ) in a polyvalent association should be similar to the cost of a monovalent interaction. In contrast, the dissociation of a polyvalent complex requires breaking many bonds therefore reducing k_{off}. The extent that polyvalent interactions are stronger than monovalent interactions will depend on the structure and geometry of the receptor and presentation of the ligands. The polyvalent association constant (K^{poly}) is described by Equation (1),

K^{poly}=(K^{mono})^{αN} (1)

wherein K^{mono} is the monovalent association constant, α is the degree of cooperativity and N is the number of bonds.

There are numerous reports of block copolymers and micelles for drug delivery. In addition, there is ample evidence in the art showing that the instant approach is efficacious. For example, surface plasmon resonance has been used previously to show that affinity targeted micelles have a 10-fold greater avidity than their monovalent equivalent. The increase in avidity for the instant conjugate compounds may be even greater since these previous studies were convoluted by a large amount of nonspecific adsorption and potentially imprecise attachment of receptors (Stella, et al. (2000) J. Pharma. Sci. 89(11):1452-1464). Micelles in general have been studied extensively as drug carriers (Kataoka, et al. (2001) Adv. Drug Deliv. Rev. 47(1):113-131; Kakizawa & Kataoka (2002) Ad. Drug Deliv. Rev. 54(2):203-222). Thermally responsive micelles have been examined, but their strategy is to first target the tumor passively through the EPR effect followed by an active temperature assisted release of encapsulated drugs (Chung, et al. (2000) J. Contr. Rel. 65(1-2):93-103). Furthermore, thermally responsive lyposomes are in clinical trials proving that thermally stimulated drug delivery can be used in the treatment of disease.

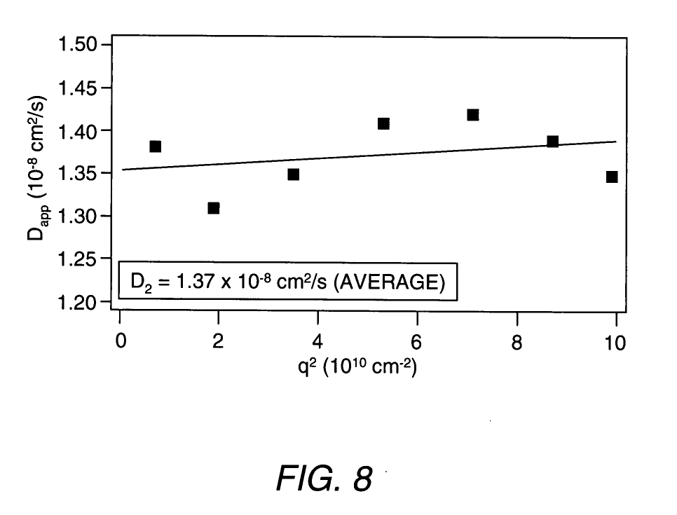

ELP-BC Vesicles. When the hydrophilic segment mass fraction is reduced, the probability of forming a lamellar phase increases. Analysis of the ELP2-32,4-90 indicates that this ELP-BC forms a structure with a hydrodynamic radius of 272 nm (FIG. 8), which static light scattering indicates is a vesicle. These ELP-BC vesicles formed in response to heat which can be used to encapsulate drugs in a similar manner as liposomes. The dissolution of these ELP-BC vesicles are easily disrupted by a further increase in temperature which can be used to release drugs at a specific site within the body.

The foregoing is illustrative of the present invention, and is not to be construed as limiting thereof. The invention is defined by the following claims, with equivalents of the claims to be included therein.

That which is claimed is:

1. A conjugate compound comprising:
(a) an active compound;
(b) an affinity binding agent; and
(c) a block copolymer, said block copolymer consisting of: (i) a first elastin-like polypeptide having a first Tₜ and (ii) a second elastin-like polypeptide having a second Tₜ greater than said first Tₜ.

3. The conjugate of claim 1, wherein said active compound is an imaging agent.

4. The conjugate of claim 1, wherein said active compound is a contrast agent.

5. The conjugate of claim 1, wherein said active compound is a therapeutic agent.

6. The conjugate claim 1, wherein said active compound is a radionuclide.

7. The conjugate of claim 1, wherein said affinity binding agent is an antibody.

8. The conjugate of claim 1, wherein said affinity binding agent is a tumor ligand.

9. The conjugate of claim 1, wherein said affinity binding agent is an antibody that selectively binds to a tumor antigen.

10. A composition comprising a conjugate of claim 1 solubilized in an aqueous carrier.

11. A method for the targeted delivering of an active compound in vivo to a selected region within a subject, comprising:
(a) administering a conjugate compound of claim 1 to said subject wherein said affinity binding agent selectively binds to said selected region;
(b) heating the selected region to a temperature greater than said first Tₜ so that the active compound is preferentially localized in said selected region.

12. The method of claim 11, wherein said subject is a mammal.

13. The method of claim 11, wherein said administering step is a systemic administering step.

14. The method of claim 11, wherein said administering step is carried out by subcutaneous injection, intraperitoneal injection, intraveneous injection, intramuscular injection, oral administration, inhalation administration, or transdermal administration.

15. The method of claim 11, wherein said selected region is a tumor.

16. The method of claim 11, wherein said heating step is carried out by application of a heat source.

17. The method of claim 11, wherein said heating step is carried out by directing radio frequency energy at said selected region.

18. A composition comprising a micelle or vesicle in a carrier, the micelle or vesicle comprising:
(a) an active compound;
(b) an affinity binding agent; and
(c) a block copolymer, said block copolymer consisting of: (i) a first elastin-like polypeptide having a first Tₜ and (ii) a second elastin-like polypeptide having a second Tₜ greater than said first T.

19. The composition of claim 18, wherein said active compound is an imaging agent.

20. The composition of claim 18, wherein said active compound is a contrast agent.

21. The composition of claim 18, wherein said active compound is a therapeutic agent.

22. The composition claim 18, wherein said active compound is a radionuclide.

23. The composition of claim 18, wherein said affinity binding agent is an antibody.

24. The composition of claim 18, wherein said affinity binding agent is an antibody that selectively binds to a tumor antigen.

25. The composition of claim 18, wherein said affinity binding agent is a tumor ligand.

26. The composition of claim 18, wherein said carrier is an aqueous carrier.

27. A method for the targeted delivering of an active compound in vivo to a selected region within a subject, comprising:
(a) administering a composition of claim 17 to said subject wherein said affinity binding agent selectively binds to said selected region;
(b) heating the selected region to a temperature greater than said first Tₜ so that the active compound is preferentially localized in said selected region.

28. The method of claim 27, wherein said subject is a mammal.

29. The method of claim 27, wherein said administering step is a systemic administering step.

30. The method of claim 27, wherein said administering step is carried out by subcutaneous injection, intraperitoneal injection, intraveneous injection, intramuscular injection, oral administration, inhalation administration, or transdermal administration.

31. The method of claim 27, wherein said selected region is a tumor.

31. The method of claim 27, wherein said heating step is carried out by application of a heat source.

32. The method of claim 27, wherein said heating step is carried out by directing radio frequency energy at said selected region.
